(19) United States
(12) Patent Application Publication
(10) Pub. No.: US 2015/0005662 A1
(43) Pub. Date: Jan. 1, 2015

(54) SYSTEM AND METHOD FOR FINE NEEDLE ASPIRATION

(71) Applicants: Robert Brik, Richmond, VA (US); Alexander Mark Lesman, Dublin, OH (US); Kevin Chen, Rego Park, NY (US); Alex Joseph Neff Speltz, San Francisco, CA (US); Jisoo Kim, Redwood City, CA (US); Alexander Slocum, Bow, NH (US); Sareh Parangi, Newton, MA (US); William Clay Faquin, Holbrook, MA (US)

(72) Inventors: Robert Brik, Richmond, VA (US); Alexander Mark Lesman, Dublin, OH (US); Kevin Chen, Rego Park, NY (US); Alex Joseph Neff Speltz, San Francisco, CA (US); Jisoo Kim, Redwood City, CA (US); Alexander Slocum, Bow, NH (US); Sareh Parangi, Newton, MA (US); William Clay Faquin, Holbrook, MA (US)

(21) Appl. No.: 14/370,850

(22) PCT Filed: Jan. 22, 2013
(86) PCT No.: PCT/US2013/022573

§ 371 Date: Jul. 7, 2014

Related U.S. Application Data
(60) Provisional application No. 61/588,891, filed on Jan. 20, 2012.

(51) Int. Cl. A61B 10/02

(52) U.S. Cl. CPC A61B 10/0266
USPC 600/566; 600/567

(57) ABSTRACT

A system and method for fine needle aspiration includes a plurality of components. A device includes a body unit, a trigger unit, and at least one gear. The body unit is configured to hold a syringe, and the trigger unit is configured to laterally move relative to the body unit in order to extend and retract a plunger within the syringe. The at least one gear is configured to engage the syringe so that lateral movement of the trigger unit causes rotation of the syringe. A needle includes a distal tip with a vertical edge extending from a distal end to a proximal end, and a curved edge running from the distal end to the proximal end. A syringe kit includes a stylet connected to the plunger. The stylet includes an outer diameter equal to an inner diameter of the needle.

### CROSS-REFERENCE TO RELATED APPLICATIONS

This application is based on, claims the benefit of, and incorporates herein by reference U.S. Provisional Patent Application Ser. No. 61/588,891, filed on Jan. 20, 2012, entitled “Toothed Needle Design and Pistol Design for Suction, Rotation, and Stylet Removal.”

### STATEMENT REGARDING FEDERALLY SPONSORED RESEARCH

N/A

### BACKGROUND OF THE INVENTION

The present application is directed to fine needle aspiration. More specifically, the present application is directed to devices and features for improving fine needle aspiration, including providing a tapered-tooth needle design, a stylet-plunger combination configured to improve tissue cell collection, and a device configured to simultaneously rotate a syringe and needle, retract the stylet through the needle, and apply suction to the needle.

Fine needle aspiration (“FNA”) is a diagnostic procedure performed to extract a cellular sample from a nodule or tumor. Commonly, FNA is used as a minimally invasive sampling technique on organs such as the thyroid, breast, lymph nodes, lung, bone, kidney, liver and pancreas. Generally, a hypodermic needle, often comprising a standard triple-grind bevel cut tip, is directed to the tissue of interest with radiologic guidance and gently moved back and forth several times to collect a sample. The sample is forced into the needle via a suction aspiration technique or a capillary aspiration technique.

For example, with regard to FNA of the thyroid, the needle, usually attached to a 10 cc syringe via a luer-lock connection, is inserted into thyroid tissue and gently moved back and forth. This allows shearing of bits of cellular tissue material from their stromal connections in the thyroid tissue and capture of these bits within a hollow body of the needle. For FNA of thyroid nodules, needles often do not exceed 21 gauge in size, and 25 to 27 gauge is preferred to reduce trauma. In the suction aspiration technique, suction is applied to the syringe in order to pull more material into the needle body. Some recent hand-held biopsy aspiration guns or pistols allow suction to be applied with one finger, thus allowing an operator to have one hand free for other uses, such as holding an ultrasound probe. In the capillary aspiration technique, no suction is applied. Rather, the needle is inserted and then rotated to and fro during back and forth oscillation, thus sheering cells into the body of the needle.

In both techniques, a sample of the biopsied material is stuck in the hollow needle body, and also often in the needle hub and the reservoir of the syringe. This material may comprise any potential tissues/cells present in the thyroid, such as red blood cells, thyroid follicular cells, stromal cells, and/or inflammatory cells. Generally, for each pass of the needle (that is, each insertion and collection), the collected cellular material is then deposited onto a glass slide and sprayed with a fixative, or directly inserted into a fixative solution, and then stained with various chemical stains. The stained material is then examined by a cyto-pathologist to diagnose benign or malignant conditions, for example by distinguishing various cellular architectural and morphological characteristics. Often, three to five passes of the needle are performed in an attempt to provide an adequate cellular yield (that is, the quality and quantity of collected tissue) for diagnosis. If the cellular yield is adequate and the cellular morphology points to a benign process in the organ, then watchful waiting is often undertaken on the mass that was biopsied. If the cells show many or all features of malignancy, then surgical excision is often recommended.

Lack of an adequate cellular yield, collection of unwanted cells (for example, blood or intervening uninformative tissues, such as fibrous tissue or muscle), and inadequate transfer of samples onto a slide can result in non-diagnostic or inconclusive needle aspirations. If the results are non-diagnostic or inconclusive, the procedure has to be repeated or more invasive biopsy methods undertaken, which increases costs and test time for patients and clinical personnel. Furthermore, in some tissues, FNA is the only biopsy tool that can be safely used. For example, FNA is the only biopsy tool used for the thyroid because other current biopsy methods, such as a standard core biopsy, are considered too dangerous to be performed given the possibility of damage to structures adjacent to the thyroid, including cerebral vessels and recurrent laryngeal nerves, due to the needle sizes currently necessary for such biopsies and the snapping motion of current core biopsy devices.

Prior attempts to improve FNA typically focus on improving either the yield or the ergonomics and ease of the procedure. For example, some attempts modify needle tip geometry with the goal of aiding tissue and cell collection in the needle body or lumen. Such attempts either modify the distal end of the needle from the standard bevel cut or maintain the bevel cut and provide one or more cut-outs near the distal end (for example, providing a “cheese-grater” design). With respect to modifying the standard beveled needle tip, which comprises a single cut along an angled plane, Franseen-type needles include a crown-point tip. Such needles are mainly designed for general tissue biopsy procedures, not commonly available in sizes smaller than 22 gauge and, as a result, are not typically used for thyroid FNA. With respect to cheese-grater designs, such needles of this style do not appear to be manufactured or commonly used in FNA procedures. Moreover, there is little clinical support on the effectiveness of this style of needle.

In addition, as discussed above, other attempts focus on improving the ease and ergonomics of the procedure. For example, one such device uses vibration to make the needle appear brightly under ultrasound, making it easier for clinical personnel to guide the needle to a desired location. In another example, some devices provide a mechanism to easily pull the syringe plunger while performing the motion of FNA (that is, oscillating the needle back and forth). In yet another example, some devices attempt to automate the FNA motion using a motor to simplify and standardize the FNA procedure. However, such powered automation devices do not appear in production and hence are not commonly used.

Although there have been many attempts, there does not appear to be a solution to the fundamental problem of low yield in cell collection, particularly in thyroid FNA. As discussed above, the solutions that do exist for improving yield are either not completely effective or only touch on a single source of problems. Furthermore, there do not appear to be devices that successfully integrate techniques for improved cell yield with a convenient and ergonomic device to allow for adoption and common use by clinical personnel.

Therefore, it would be desirable to provide an ergonomic and easy-to-use device that improves the diagnostic cellular yield and accuracy of fine needle aspirations.

### SUMMARY OF THE INVENTION

The present invention overcomes the aforementioned drawbacks by providing a number of features for improving fine needle aspiration, including a device, a needle tip design, and a stylet configuration. These features, collectively or individually, can improve uptake of tissue and cells, deter reduction of sample quality due to coagulation of blood, and/or reduce quantity of sample lost during transfer to a slide in comparison to traditional fine needle aspiration techniques. Furthermore, the device of the present invention provides improved accuracy and ease of use during fine needle aspiration procedures.

Thus, in accordance with one aspect of the invention, a device configured for use with a syringe including a plunger and a needle for fine needle aspiration includes a body unit, a trigger unit, and at least one gear. The body unit is configured to hold the syringe, and the trigger unit is configured to engage the plunger and to laterally move relative to the body unit to extend and retract the plunger within the syringe. The at least one gear is configured to engage the trigger unit and to rotate in response to lateral movement of the trigger unit. Furthermore, the at least one gear is configured to engage the syringe to cause rotation of the syringe responsive to lateral movement of the trigger unit.

In accordance with another aspect of the invention, a needle for fine needle aspiration includes a hollow needle body running along an axis with a proximal tip and a distal tip. The distal tip includes a vertical edge extending parallel to the axis from a distal end located at the distal tip to a proximal end located a length displaced from the distal tip toward the proximal tip. The distal tip also includes a curved edge running along a portion of the distal tip from the distal end to the proximal end.

In accordance with yet another aspect of the invention, a syringe kit for use with a needle in fine needle aspiration includes a syringe with a distal end configured to connect to the needle, a plunger configured to move within the syringe between an extended position and a retracted position, and a stylet connected to the plunger and configured to extend through at least a portion of the distal end into the needle when the plunger is in the extended position and the retracted position. The stylet includes an outer diameter substantially equal to an inner diameter of the needle to substantially prevent material collected within the needle past a distal tip of the stylet.

These and other features and advantages of the present invention will become apparent upon reading the following detailed description when taken in conjunction with the drawings.

### BRIEF DESCRIPTION OF THE DRAWINGS

FIG. 1 is a partial perspective view of a needle including a double tapered-tooth tip according to the present invention;

FIG. 2 is a partial perspective view of a needle including a single tapered-tooth tip according to the present invention;

FIG. 3a is a cross-sectional view of a needle connected to a plunger, including aspects of the present invention, in an extended position;

FIG. 3b is a partial cross-sectional view of the needle of FIG. 3a;

FIG. 4a is a cross-sectional view of a needle, including aspects of the present invention, in a retracted position;

FIG. 4b is a partial cross-sectional view of the needle of FIG. 4a;

FIG. 5 is a partial perspective view of a needle and a stylet of the present invention;

FIGS. 6A-6C are perspective, partial perspective, and cross-sectional views, respectively, of a device, according to the present invention, in an extended position;

FIGS. 7A-7C are perspective, partial perspective, and cross-sectional views, respectively, of a device, according to the present invention, in a retracted position;

FIG. 8 is a partial front cross-sectional view of the device of FIGS. 6A-7C;

FIG. 9 is a partial side cross-sectional view of the device of FIGS. 6A-7C; and

FIG. 10 is a perspective view of another device according to the present invention.

### DETAILED DESCRIPTION OF THE INVENTION

Generally, in fine needle aspiration (“FNA”), a needle coupled to a syringe is inserted into a tissue of interest and gently moved back and forth. This allows shearing of bits of cellular tissue material and capture of these bits within the needle body. Suction can be applied to the syringe in order to pull more cells into the needle body. Also, the needle can be rotated to further shear cells and facilitate sample collection within the needle body. After collection and removal of the needle from the tissue of interest, the collection material must be extracted from the needle onto a slide for examination. The present invention provides a number of features for improving FNA, including a device, a needle tip design, and a stylet configuration. These components provide improved diagnostic cellular yield as well as improved accuracy and ease of use during FNA procedures. As further described below, more accurate diagnostic sampling can result in decreased excisional biopsies, as well as a decrease in removal of whole organs.

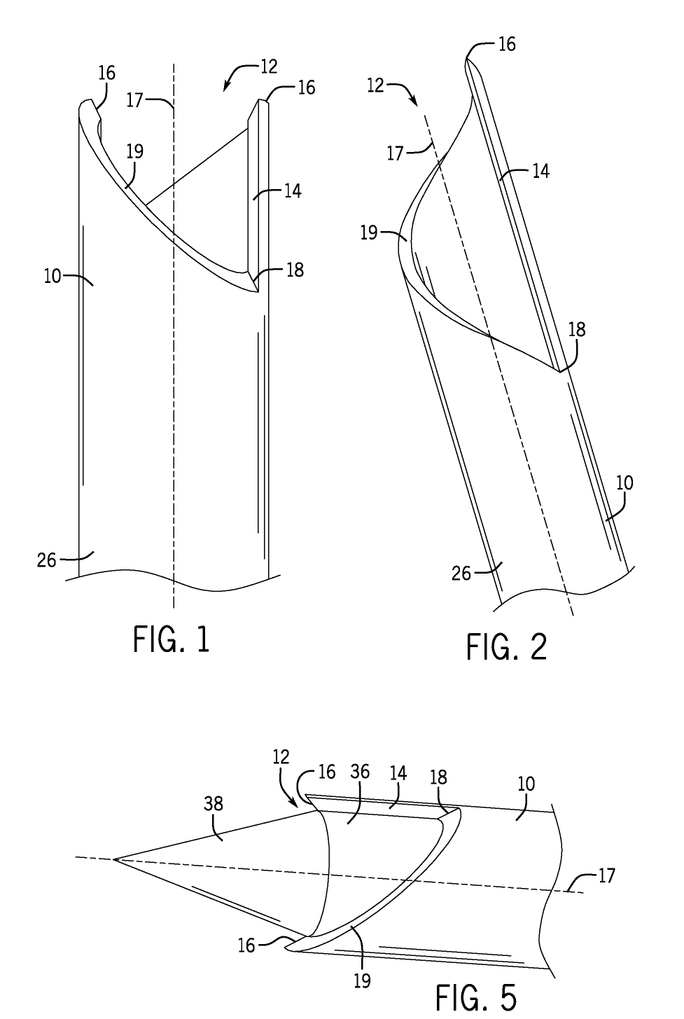

With respect to needle designs of the present invention, FIGS. 1 and 2 illustrate a FNA needle 10 with a distal, tapered-tooth needle tip 12 (double-tooth and single-tooth, respectively). These tapered tooth tips 12 can improve uptake of tissue and cells in comparison to traditional FNA needle designs. To accomplish such improvements, each tip 12 includes sharp vertical edges 14 (that is, parallel to the needle 12), as shown in FIGS. 1 and 2. More specifically, the two edges 14 of the double-tooth design, or the single edge 14 of the single-tooth design, can encounter tissue during oscillation of the tapered-tooth needle 10, shears cells from the tissues, and guide them into the needle 10.

As shown in FIGS. 1 and 2, the length of the needle 10 (for example, of a body 26 of the needle 10) lies along an axis 17. Each vertical edge 14 can extend parallel to the axis 17 and can be defined between a distal end 16, located at the distal tip 12, and a proximal end 18, located some length displaced from the distal tip 12. As a result, a curved edge 19 defines a gradual reduction in length around at least a portion of the needle tip 12 from one distal end 16 to an adjacent proximal end 18, for example in comparison to one or more diagonal cuts of traditional bevel or franseen-type needle tips. In other words, bevel and franseen-type needles include full-plane cuts across the entire needle tip, while the tapered-tooth designs of the present invention only cut partially into the needle tip along any given plane (for example, one cut along a plane into half the needle tip 12, and then another cut along a mirrored plane into the other half of the needle tip 12).

Furthermore, bevel needles are designed to purposely cause tissue to separate along a single plane into two pieces and move around the needle. Since bevel tips were primarily designed for injections, this was a favorable feature because it helps prevent clogging of tissue within the needle. The goal of FNA, however, is to collect material within the needle. In the double-tooth design, the distal ends 16 are offset in a way to create a corkscrew action. Due to the offset and its resulting corkscrew effect, the tapered tooth design can avoid shearing along a single plane, thus preventing tissue from deflecting around the needle 10 and allowing collection of more cells as well as larger clusters of cells or intact tissue. More specifically, the tapered tooth design cuts into tissues at the number of points equal to the number of teeth (for example, one or two) and better digs into the tissue, rather than separating the tissue, due to the slope of the teeth.

In some implementations, for example for thyroid FNA, the needle 10 can be sized at 25 gauge or 27 gauge. Smaller or larger needle sizes are also contemplated in accordance with the present invention, ranging from, for example, 29 gauge to 21 gauge. Furthermore, the tapered tooth designs, while improving cellular yield, do not impede or reduce an inner diameter of the needle 10. As a result, smaller needle sizes may be used in some FNA procedures (that is, to retrieve the same cellular yield that previously required a larger needle). For example, as a result of the unimpeded inner diameter, the needle designs of the present invention can allow removal of a tissue core, similar to that retrieved with a core biopsy, to permit a more detailed cytologic and histologic evaluation. Accordingly, core biopsies, which are currently not used for thyroid FNA due to needle size requirements, can be performed using the needle 10 of the present invention.

The needle tips 12 can also be designed based on the type of collection desired. For example, in some cases, as described above, a core biopsy may be desired to retrieve a large chunk of material. In other cases, a variety of cells is desired, instead of a large core. It is contemplated that the balance between a greater variety of cells and a more core biopsy-type result is dependent upon the cut angles along the needle tips 12. Accordingly, needle tips 12 with sharper cut angles can allow for a core biopsy, while needle tips 12 with shorter cut angles (that is, stubbier needle tips 12) can retrieve a wider variety of cells.

In addition, in some implementations, the needle 10 can be constructed of stainless steel to allow for visibility through ultrasound. The needle 10 can also include outer threading, serrations, or other texture (not shown) to enhance clarity of the needle 12 during ultrasound and/or to improve insertion toward the tissue of interest. Furthermore, in some implementations, the needle 12 can include additional features, such as internal threading.

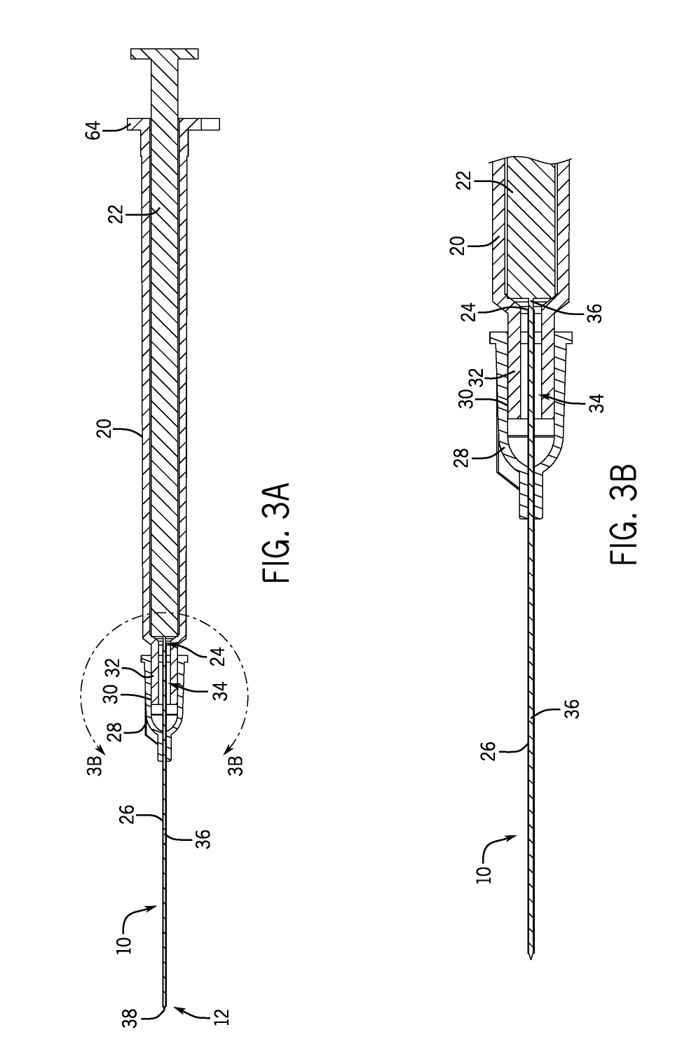

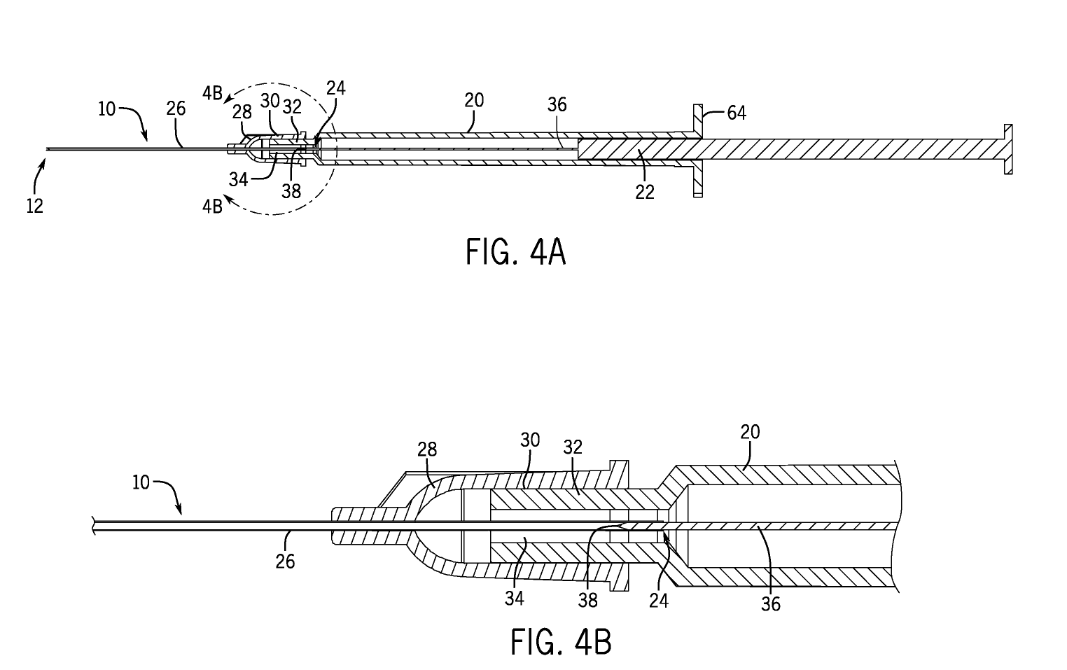

FIGS. 3A-3B and 4A-4B illustrate a needle 10 of the present invention coupled to a syringe 20 with a plunger 22 in an extended state, where the plunger 22 is pressed fully into the syringe, and a retracted state, where the plunger 22 is retracted outward from the syringe 22, respectively. As shown in FIGS. 3A-4B, the needle 10 can include the distal needle tip 12, as described above, a proximal tip 24, a hollow needle body 26, and a hub 28. The needle 10 can be coupled to the syringe 20 (such as a 10 cubic centimeter syringe), for example, through a female fitting connection 30 of the hub 28 and a male fitting connection 32 of the syringe 20 (such as luer-lock fitting connections). In order to reduce sample loss, the present invention can allow for the needle 10 to keep the collected sample within the needle body 26 at all times, instead of allowing sample to exit from the proximal tip 24 into the syringe 20 or the needle hub 28. For example, an internal volume of the needle 10 can be increased, in comparison to conventional designs, by extending the needle body 26 into a syringe hub 34 (defined by the male fitting connection 32), and/or using extra thin wall needle tubing. By preventing the sample from collecting in the syringe, this extended-needle design can deter reduction of sample quality due to coagulation of blood and also reduce a quantity of sample lost during transfer to the slide. In some cases, such as for thyroid FNA, the needle 10 can be long enough to extend into or through the syringe hub 34 and also permit an insertion depth, or needle length extending out from the syringe 20, greater than about 1.5 inches.

In addition, the syringe 20 can incorporate a stylet 36 configured to extend through the length of the needle 10, as best shown in FIGS. 3B and 4B. The stylet 36 can provide structural support to help prevent the needle 10 from deflecting or breaking when being inserted into a tissue, thus making the needle 10 safer and easier to guide than traditional hollow needles. The stylet 36 can also restrict a collected sample from escaping the proximal tip 24 of the needle 10 into the syringe 20 or the needle hub 28, as further described below. By preventing the sample from collecting in the syringe 20, the stylet 36 can deter reduction of sample quality due to coagulation of blood and also reduce a quantity of sample lost during transfer to the slide.

According to some aspects of the present invention, the stylet 36 can include a diameter less than or equal to an inner diameter of the needle 10 to facilitate back and forth movement through the needle body 26. For example, in an extended position, a tip 38 of the stylet 36 can extend outward past the distal tip 12 of the needle 10, as shown in FIGS. 3A and 5. The stylet 36 can be constructed of a substantially rigid material, such as carbon steel, to provide structural support when in the extended position, for example during needle insertion into a tissue. Furthermore, as best shown in FIG. 5, the stylet tip 38 can be substantially sharp and conical in shape. The conical tip 38 can minimize deflection of the needle's distal tip 12 and help reduce insertion force of the needle 10 into a tissue. In addition, because the stylet 36, when extended, protrudes past the distal tip 12 of the needle 10, the stylet 36 can prevent muscle, skin and other undesirable tissue from clogging the needle body 26 as it is guided to the tissue or nodule of interest, thus improving the quality of a collected sample.

The stylet 36 can also be retracted away from the distal tip 12 of the needle 10 until it reaches a fully retracted position, as shown in FIGS. 4A-4B. In this fully retracted position, the stylet 36 can remain partially within the needle body 26, as best shown in FIG. 4B, thus preventing any collected sample from escaping the needle body 26 and becoming stuck in the needle hub 28 and/or the syringe 20. Furthermore, after retraction of the stylet 36, sample collection, and extraction of the needle 10 from the tissue of interest, the stylet 36 can again be extended to help push the collected material out of the needle body 26 and onto a slide. Because the diameter of the stylet 36 can match the inner diameter of the needle body 26, the sample is substantially prevented from traveling past the stylet tip 38 and, thus, most, if not all, of the collected sample can be forced out of the needle body 26 by extending the stylet 36. Furthermore, in order to prevent a sample from traveling past the stylet tip 38 both the diameter of the stylet 36 and the inner diameter of the needle body 26 can remain constant (that is, do not increase or decrease) along their respective lengths.

In some implementations, as shown in FIGS. 3A-4B, the stylet 36 can be rigidly connected to a distal end of the plunger 22 so that movement of the plunger 22 causes movement of the stylet 36 through the needle body 26. For example, after the needle 10 and the stylet 36 are inserted into a tissue, the plunger 22 can be retracted in order to both retract the stylet 36 and provide suction within the needle body 26 to cellular material. In some implementations, the syringe 20, with connected plunger 22 and stylet 36, can be provided as a specialized FNA syringe kit.

In addition, in some implementations, the stylet 36 can be replaced with or incorporate an extendable brush (not shown). Egress of the brush from the needle tip 12 and subsequent rotation of the brush can allow further capture of cells and tissue clusters onto the brush. The brush can then be retracted back into the needle body 26 during removal of the needle 10 from the tissue. This type of cytologic brushing is performed in some tubular structures such as pancreatic ducts, cystic ducts, and ureters, but has never been described for use in biopsy of a solid organ, such as the thyroid.

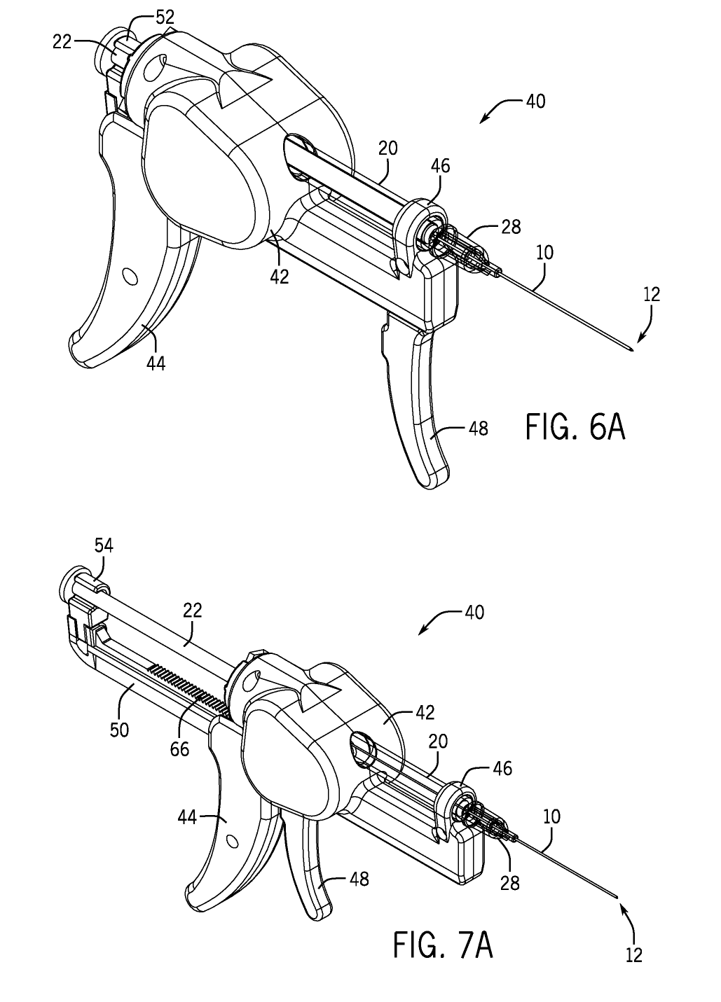

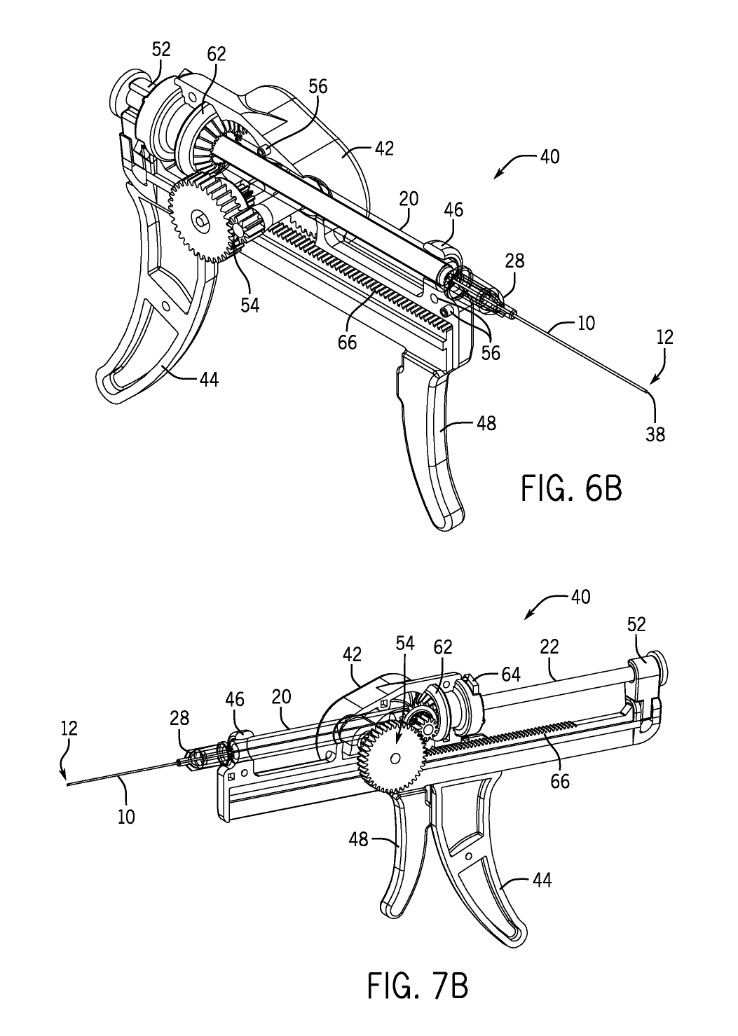

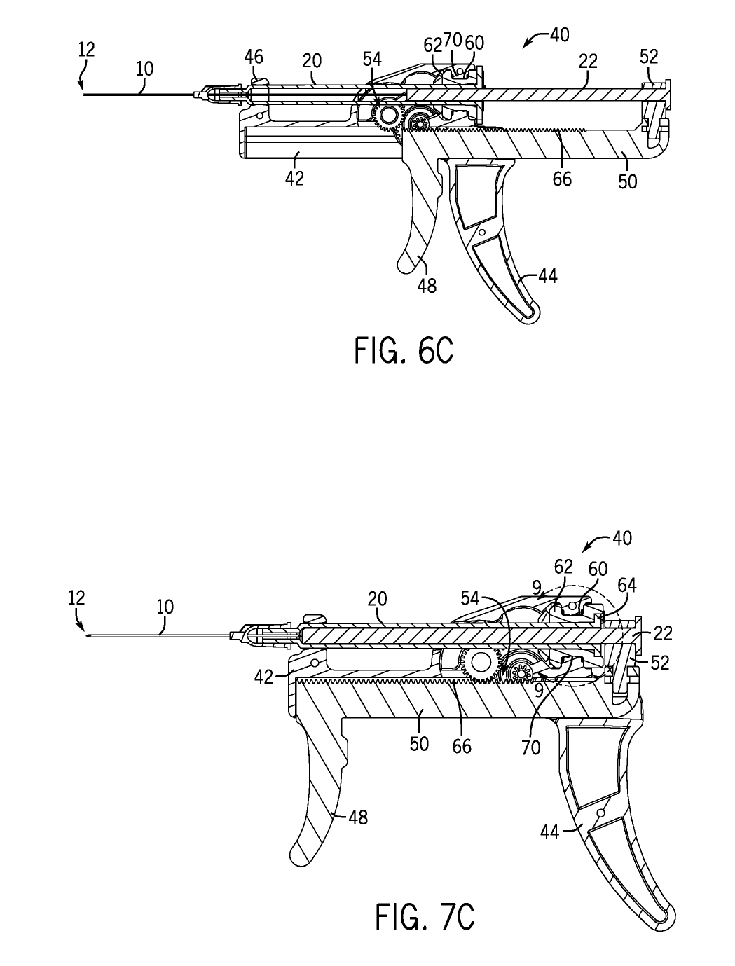

In addition to the needle 10 and stylet 36 described above, aspects of the present invention provide an FNA device 40, as shown in FIGS. 6A-9. The device 40 can be a hand-held, pistol-type device for single-handed use by an operator (such as a physician or clinical personnel). The device 40 can be configured for use with standard syringes and needles, or with the needle 10 and the stylet 36 of the present invention. Accordingly, the device 40 can be configured to hold a syringe 20, including a plunger 22 and connected stylet 36. The device 40 can be adjusted to a fully extracted position, as shown in FIGS. 6A-6C, for example during insertion of the needle 10 into a tissue of interest and during expulsion of collected cells onto a slide. Furthermore, the device 40 can be adjusted to a fully retracted position, as shown in FIGS. 7A-7C, for example during sample collection and during withdrawal of the needle 10 from the tissue of interest after collection.

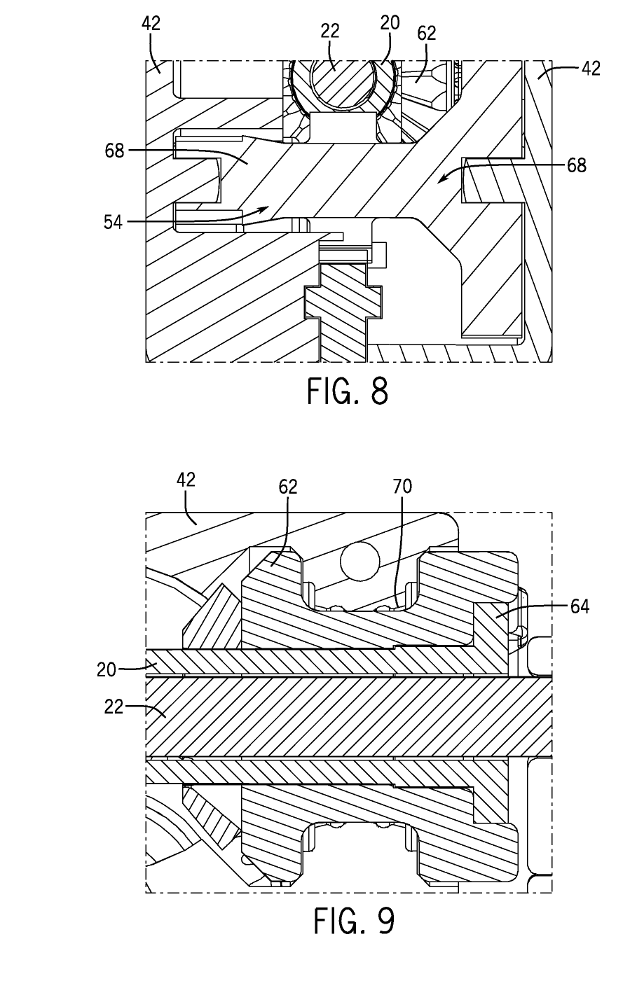

As shown in FIGS. 6A-7C, the device 40 can include a body 42, a grip portion 44, one or more syringe guides 46, a trigger 48, a sled 50, a plunger clip 52, and a series of gears 54 (substantially enclosed by the body 42). The body 42, the grip portion 44, and/or the syringe guides 46 can be formed as an integral unit or otherwise coupled together, thus forming a “body unit.” Furthermore, in some implementations, the body unit can be formed as two halves capable of being temporarily coupled together (via snap features, screws, etc.) or permanently coupled together (via glue, bonding, etc.). For example, FIGS. 6B, 6C, 7B, and 7C illustrate corresponding snap features 56 and each body unit half. The body unit can also include bearings 58, shown in FIG. 8, on internal surfaces of each half to support at least some of the gears 54. In addition, the trigger 48, the sled 50, and/or the plunger clip 52 can be formed as an integral unit or otherwise coupled together, thus forming a “trigger unit.”. For example, FIGS. 6B, 6C, 7B, and 7C illustrate the trigger 48 and the sled 50 as an integral piece and the plunger clip 52 coupled to a proximal end of the sled 50 via a hinged connection.

The syringe 20 can be inserted through the syringe guide 46 and an internal aperture 60 of a main gear 62, for example so that the syringe flange 64 abuts a proximal side of the main gear 62, and the plunger 22 can be adjusted so that it rests within the plunger clip 52. In order to ease syringe insertion and removal, the plunger clip 52 can be moved or folded out of the way, due to the hinged connection with the sled 50, during insertion and removal and then clipped back into place during use in order to engage the plunger 22. The syringe guide 46 and the plunger clip 52 can be sized to allow free rotation of the syringe 20 and the plunger 22, respectively, while the internal aperture 60 of the main gear 62 can be sized so that rotation of the main gear 62 causes rotation of the syringe 20, as further described below.

In use, the device 40 is configured to retract the plunger 22, retract the stylet 36, and rotate the syringe 20 and the needle 10 upon actuation of the trigger 48. More specifically, actuation, or pull-pack, of the trigger 48 (that is, toward the grip portion 44) causes retraction of the plunger 22 through the syringe 20 (via pull-back of the plunger clip 52) as well as retraction of the connected stylet 36. Furthermore, as shown in FIGS. 6B-7C, the body unit can form an internal track upon with the sled 50 can move (for example, between a distal end of the body 42 and the grip portion 44). The sled 50 can include a threaded portion 66 configured to engage at least one of the gears 54 so that actuation of the trigger 48 and resultant linear movement of the sled 50 causes the gears 54 to rotate. More specifically, the gears 54 are arranged in a multi-stage fashion so that linear movement of the sled 50 causes rotation of the main gear 62 and, thus, rotation of the syringe 20, the connected stylet 36, and the connected needle 10.

Accordingly, during an FNA procedure, an operator can use the device 40, in a fully extended position, to insert the needle 10 into a tissue of interest. As the device 40 is configured for single-handed use, the operator is capable of using their other hand to maneuver an ultrasound probe to assist with guiding the needle 10. Upon positioning the needle 10 in the tissue of interest, the operator can oscillate the needle 10 back and forth within the tissue while simultaneously actuating the trigger 48, causing retraction of the stylet 36, suction through the needle body 26, and rotation of the needle 10 to enhance sample collection within the needle body 26.

More specifically, as the trigger 48 is moved backward toward the grip portion 44, the stylet 36, linked to the plunger 22, is pulled toward the proximal tip 24 of the needle 10. As a result, the distal tip 12 of the needle 10 becomes exposed and is able to dig into the tissue. As described above, the needle 10 can be long enough to extend into or through the syringe hub 34, thus preventing the stylet 36 from fully exiting the needle body 26. The stylet 36 therefore seals the proximal tip 24 of the needle 10 and prevents material from moving into the needle hub 28 or the syringe 20. In addition to retracting the stylet 36, the backward movement of the plunger 22 creates suction in the needle body 26 to pull up the desired cellular material. Meanwhile, the moving sled 50 causes the gears 54, and thus the needle 10 and the syringe 20, to rotate, as described above, further aiding in material collection.

Once trigger actuation is complete, while in the fully retracted position, the operator can remove the needle 10 from the tissue. In this fully retracted position, suction, or negative pressure, is still provided within the needle body 26 therefore containing the material within the needle body 26 and preventing the spread of collected, potentially cancerous, material during needle travel. Once the needle 10 is removed, the collected contents must be transferred to a slide or other liquid fixative. The trigger unit can easily be moved back into its fully extended position by pushing the trigger 48 or the proximal end of the sled 50 near the plunger clip 52, causing forward movement of both the plunger 22 and the connected stylet 36. The contents are expelled due to a combination of pressure force from the plunger 22 and the stylet 36 physically ejecting the material.

The entire FNA process including, for example, insertion, collection, removal, and ejection onto slide, can be performed in less than about one minute, or less than about two minutes. In some implementations, actuation of the trigger 48 from the fully extended position to the fully retracted position (for example, when the trigger 48 has been actuated its full distance and abuts the grip portion 44) can cause about 30 to 60 rotations of the needle 10. The number of rotations can also be adjusted through different gear designs. For example, in another implementation, the trigger 48 can be actuated with about 12 Newtons of force to provide about 15 or more rotations. Furthermore, in some implementations, each needle pass can collect about 150 to about 200 or more cells within the needle body 26 using the needle 10, the stylet 36, and the device 40 of the present invention. This collection amount can also be adjusted to accommodate specific FNA requirements or preferences.

The design of FIGS. 6A-7C includes five gears 54, including the main gear 62, to accomplish rotation of the syringe 20 through actuation of the trigger 48. However, other implementations of the present invention can provide more or less gears or other gear-based mechanisms to accomplish this movement. Design considerations for such gear-based mechanisms include providing proper parts strength and minimizing frictional losses. For example, due to the size of the gears 54, design considerations include appropriately supporting the gears 54 without introducing issues with regard to the axle diameter to axle length ratio, as well as bearing surface diameter to body diameter ratio. Further considerations also include supporting ends of the internal components to prevent them from sliding on the axles. The friction at these end supports can be reduced through the use of domed axles 68, as shown in FIG. 8. Another consideration with respect to friction concerns the main gear 62. This component is supported externally from the body 42, resulting in a larger diameter bearing surface. Additionally, this component experiences the most external forces due to needle insertion and plunger retraction. For these reasons, a bearing surface 70 for the main gear 62, as shown in FIG. 9, can be designed to capture the main gear 62 in a way that minimizes normal forces and provide suitable lubrication. In some implementations, ball bearings (not shown) can be incorporated to reduce friction of the rotating gears 54.

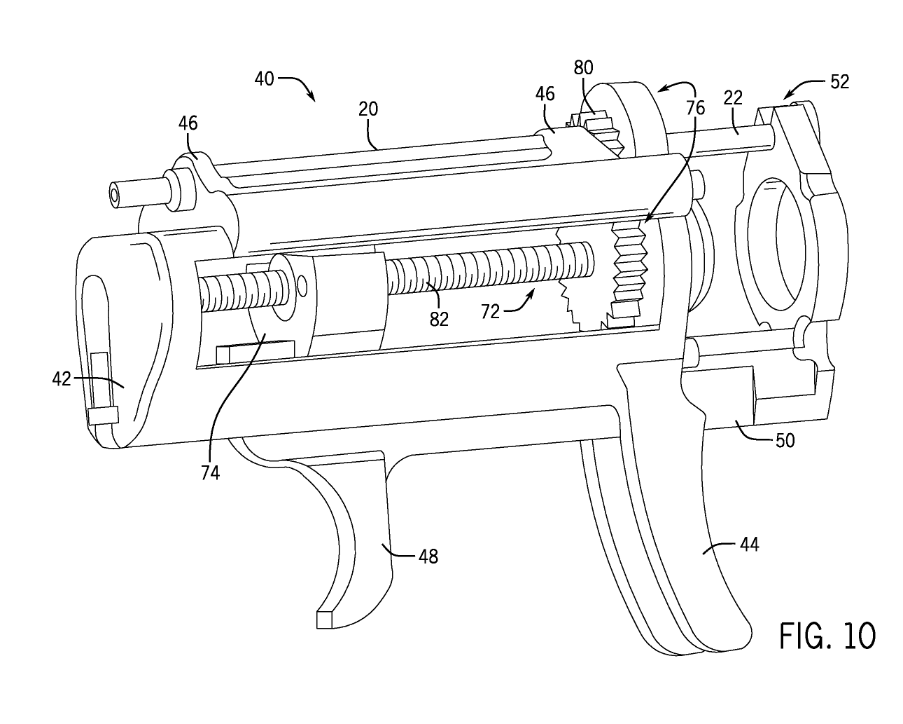

FIG. 10 illustrates a device 40 according to another implementation of the invention. The device 40 of FIG. 10 can be similar to the device 40 of FIGS. 6A-9, but include a threaded-rod based system instead of a gear-based system. Accordingly, the device 40 of FIG. 10 can include a body 42, a grip portion 44, one or more syringe guides 46, a slidable trigger 48, a sled 50, a plunger clip 52, a threaded rod or lead screw 72, a screw nut 74, and meshing gears 76. In some implementations, the body 42, the grip portion 44, and the syringe guides 46 are formed as an integral unit or otherwise coupled together, thus formatting a “body unit,”, and the trigger 48, the sled 50, the screw nut 74, and the plunger clip 52 are formed as an integral unit or otherwise coupled together, thus forming a “trigger unit.”

The body can form an internal track upon with the sled 50 can slide, causing movement of the plunger clip 52 and the screw nut 74 relative to the body unit. In addition, in some implementations, the body unit can be constructed as two halves capable of being temporarily coupled together (via snap features, screws, etc.) or permanently coupled together (via glue, bonding, etc.). The body unit may also substantially cover or enclose the screw nut 74, the lead screw 72, and/or the meshing gears 76 in some implementations.

The device 40 can be configured for use with standard syringes and needles, or with the needle 10 and the stylet 36 of the present invention. Similar to that described above with respect to FIGS. 6A-9, a syringe 20 (with plunger 22 and connected stylet 36) can be inserted through the syringe guides 46 and an internal aperture (not shown) of a first meshing gear 80, for example so that the syringe flange (not shown) abuts a proximal side of the first meshing gear 80, and the plunger 22 can be adjusted so that it rests within the plunger clip 52. The syringe guides 46 and the plunger clip 52 can be sized to allow free rotation of the syringe 20 and the plunger 22, respectively, while the internal aperture 78 of the meshing gear 80 can be sized so that rotation of the meshing gear 80 causes rotation of the syringe 20.

In use, the device 40 is configured to retract the plunger 22, retract the stylet 36, and rotate the syringe 20 and needle 10 upon actuation of the trigger 48. More specifically, actuation of the trigger 48 causes retraction of the plunger 22 from the syringe 20 (via pull-back of the plunger clip 52) and linear movement of the screw nut 74 along the lead screw 72, thus causing rotation of the lead screw 72 (that is, due to internal threading of the screw nut 74, not shown, engaging threading 82 of the lead screw 72). As discussed above, retraction of the plunger 22 causes retraction of the stylet 36 within the needle body 26. Furthermore, rotation of the lead screw 72 is then multiplied by the meshing gears 76 to cause rotation of the syringe 20. The combination of actions, all powered by actuating the trigger 48, causes the needle 10 to dig into the tissue through rotation, while suction simultaneously pulls material into the needle body 26.

The design of FIG. 10 includes two meshing gears 76 as well as the screw nut 74 and the lead screw 72 to accomplish rotation of the syringe 20 through actuation of the trigger 48. However, other implementations of the present invention can provide more or less gears or other threaded rod-based mechanisms to accomplish this movement. In some implementations, the screw nut 74 can be a self-lubricating acetyl nut. Also, thrust bearings (not shown) can be positioned along either end of the lead screw 72 to avoid screw nut displacement and binding.

With respect to both devices 40 described above, the device length, the grip portion 44, and the trigger 48 can be sized for operator comfort and ease of use. The ergonomic pistol design can also improve needle control during insertion, collection, and removal. Furthermore, some or all parts of each device 40 can be constructed of injection-molded plastic. It is also contemplated that, for increased strength and/or durability, some of the gears 54, 76 and their accompanying pins or bearings may be metal (such as steel or brass). In addition, either device 40 can be sterilized for multiple uses or can be single-use, disposable devices. Accordingly, in some implementations, as described above, both the needle 10 and the syringe 20 can be easily removed and replaced, allowing easy reuse of either device 40. In other implementations, if the device 40 is disposable after a single use, the syringe 20, the plunger 22, and the needle 10 can be built into the device 40 as a single, integral unit. Also, according to some implementations of the invention, syringe-shaped devices (that is, rather than pistol-shaped devices 40, as described above) may incorporate any or all of the features described above.

The above-described aspects of the present invention can be use singularly or collectively to improve FNA in patients with all manners of tumors, including those with thyroid breast, kidney, pancreas, lung, bone and lymph node neoplasms. For example, results of tests comparing the needle 10 of the present invention to conventional bevel-tip needles during a traditional FNA procedure illustrated that the needle 10 of the present invention is capable of significantly increasing an amount of collected cells. Results of tests comparing use of the conventional bevel-tip needle during a traditional FNA procedure to use of the conventional bevel-tip needle with the device 40 of the present invention illustrated that use of the device 40 also increased an amount and quality of collected cells. Both results thus indicate that aspects of the present invention can improve cellular yield (in both quantity and quality) when compared to traditional components and procedures. Based on these results, aspects of the present invention can increase diagnostic precision, reduce the number of inconclusive or non diagnostic aspirations, reduce needle size in some biopsy procedures and/or reduce unnecessary surgical procedures for patients. Accordingly, aspects of the present invention can have an immediate and significant impact on how confidently clinicians are able to tell benign from malignant growths in patients and, thus, save time and cost for diagnosis.

The present invention has been described in terms of one or more preferred embodiments, and it should be appreciated that many equivalents, alternatives, variations, and modifications, aside from those expressly stated, are possible and within the scope of the invention.

1. A device configured for use with a syringe including a plunger and a needle for fine needle aspiration, the device comprising:
a body unit configured to hold the syringe;
a trigger unit configured to engage the plunger and to laterally move relative to the body unit to extend and retract the plunger within the syringe; and
at least one gear configured to engage the trigger unit and rotate in response to lateral movement of the trigger unit, the at least one gear configured to engage the syringe to cause rotation of the syringe responsive to lateral movement of the trigger unit.

2. The device of claim 1, wherein the body unit includes a body, a grip portion, and a syringe holder, and the trigger unit includes a trigger, a sled, and a plunger clip.

3. The device of claim 2, wherein the plunger clip is configured to engage the plunger and is coupled to the sled by a hinged connection.

4. The device of claim 2, wherein the sled includes a threaded portion configured to engage the at least one gear.

5. The device of claim 4, wherein the at least one gear includes a first gear and a second gear, wherein the first gear engages the threaded portion and the second gear engages the syringe.

6. The device of claim 2, wherein the body encloses the at least one gear.

7. The device of claim 2 and further comprising a threaded rod coupled to the at least one gear and a screw nut coupled to the sled, wherein the screw nut is configured to laterally move with the trigger unit, wherein lateral movement of the screw nut causes rotation of the threaded rod and the at least one gear.

8. The device of claim 2, wherein the body forms a track portion and the sled is configured to laterally move along the track portion.

9. The device of claim 2, wherein the trigger is positioned relative to the grip portion to allow a single-handed pistol grip by a user.

10. The device of claim 1, wherein the body unit comprises two halves coupled together by mating snap features on each of the two halves.

11. A needle for fine needle aspiration, the needle comprising:
a hollow needle body extending along an axis;
a proximal tip at a first end of the hollow needle body; and
a distal tip at a second end of the hollow needle body opposite the first end, the distal tip including:
a vertical edge extending parallel to the axis from a distal end located at the distal tip to a proximal end located a length displaced from the distal tip toward the proximal tip, and
a curved edge extending along a portion of the distal tip from the distal end to the proximal end.

12. A syringe kit for use with a needle in fine needle aspiration, the syringe kit comprising:
a syringe including a distal end configured to connect to the needle;
a plunger configured to move within the syringe between an extended position and a retracted position, and
a stylet connected to the plunger and configured to extend through at least a portion of the distal end into the needle when the plunger is in the extended position and the retracted position,
the stylet comprising an outer diameter substantially equal to an inner diameter of the needle to substantially prevent material collected within the needle past a distal tip of the stylet.

13. The syringe kit of claim 12, wherein the distal tip of the stylet is configured to extend past a distal tip of the needle when the plunger is in the extended position.

14. The syringe kit of claim 13, wherein the distal tip of the stylet comprises a pointed, conical shape.

15. The syringe kit of claim 13, wherein the distal tip of the stylet is configured to be positioned between the distal tip of the needle and a proximal tip of the needle when the plunger is in the retracted position.

16. The syringe kit of claim 12 further comprising a needle for fine needle aspiration, the needle comprising:
a hollow needle body extending along an axis;
a proximal tip at a first end of the hollow needle body; and
a distal tip at a second end of the hollow needle body opposite the first end, the distal tip including:
a vertical edge extending parallel to the axis from a distal end located at the distal tip to a proximal end located a length displaced from the distal tip toward the proximal tip, and
a curved edge extending along a portion of the distal tip from the distal end to the proximal end.

17. The syringe kit of claim 12 further comprising a device configured for use with the syringe, plunger, needle, the device comprising:
a body unit configured to hold the syringe;
a trigger unit configured to engage the plunger and to laterally move relative to the body unit to extend and retract the plunger within the syringe; and
at least one gear configured to engage the trigger unit and rotate in response to lateral movement of the trigger unit, the at least one gear configured to engage the syringe to cause rotation of the syringe responsive to lateral movement of the trigger unit.
